(19) United States
(12) Patent Application Publication
(10) Pub. No.: US 2024/0081939 A1
(43) Pub. Date: Mar. 14, 2024

(54) CONSTRUCTING A 3D PHANTOM WITH A MATRIX MATERIAL HAVING CONDUCTIVE PARTICLES DISPERSED THEREIN

(71) Applicant: Novocure GmbH, Root D4 (CH)

(72) Inventors: Yoram Wasserman, Haifa (IL); Stas OBUCHOVSKY, Haifa (IL); Nataliya KUPLENNIK, Haifa (IL); David SHAPIRO, Haifa (IL)

(21) Appl. No.: 18/463,887

(22) Filed: Sep. 8, 2023

Related U.S. Application Data
(60) Provisional application No. 63/374,986, filed on Sep. 8, 2022.

(51) Int. Cl. A61B 90/00; A61B 34/10; A61N 1/40

(52) U.S. Cl. CPC A61B 90/08; A61B 34/10; A61N 1/40; A61B 2034/104; A61B 2090/0818; A61B 2562/046

(57) ABSTRACT

A system and method for constructing a 3D phantom with epoxy utilizing conductive particles is herein disclosed. The phantom, comprising: a container for housing a fluid, the container having at least one exterior wall comprising an exterior surface and an interior surface defining a cavity, the exterior wall constructed of a non-conductive matrix material mixed with conductive particles to provide the exterior wall with an electrical impedance, resistance, resistivity, or conductivity to model electrical impedance, resistance, resistivity, or conductivity of a first biological component; and a conductive solution within the cavity, the conductive solution being at least one of a fluid solution, a suspension, and a gel and configured to model electrical impedance, resistance, resistivity, or conductivity of a second biological component.

### REFERENCE TO RELATED APPLICATIONS

The present patent application claims priority to the provisional patent application filed on Sep. 8, 2022 and identified by U.S. Ser. No. 63/374,986, the entire contents of which are hereby expressly incorporated herein by reference.

### STATEMENT REGARDING FEDERALLY SPONSORED RESEARCH OR DEVELOPMENT

Not Applicable.

### BACKGROUND

Tumor Treating Fields (TTFields or TTFs) are low intensity (e.g., 1-3 V/cm) alternating electric fields within the intermediate frequency range (50 kHz to 1 MHz, such as, for example, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the transducer arrays that make up each of these pairs are positioned on opposite sides of the body part that is being treated. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of non-conductive ceramic disk electrodes, which are coupled to the patient's skin (such as, but not limited to, the patient's shaved head for treatment of GBM) through a layer of conductive medical gel. To form the ceramic disk electrodes, a conductive layer is formed on a top surface of nonconductive ceramic material. A bottom surface of the nonconductive ceramic material is coupled to the conductive medical gel.

One approach to applying the TTField in different directions is to apply the field between a first set of electrodes in a first direction for a period of time, then applying a field between a second set of electrodes in a second direction for a period of time, then repeating that cycle for an extended duration (e.g., over a period of days, weeks, or months). In order to generate the TTFields, current is applied to each electrode of the transducer array.

### SUMMARY OF THE DISCLOSURE

The TTFields interact with the patient and one or more of the patient's organs based on the electrical impedance, resistance, resistivity, or conductivity of each of the patient's organs. As the TTField interacts with the patient, the field may change shape based in part on the electrical impedance, resistance, resistivity, or conductivity and relative position of each of the patient's organs. Because the electrical conductivity of each organ of a patient modifies the TTField shape and a particular TTField shape may be needed to effectively target a tumor, it is important to be able to determine how the applied TTField is shaped within a patient.

To date, there has not been a way to measure actual TTField shape in a patient, without computer simulations; however, computer simulations or other models are reliant on programming techniques and estimations, and cannot show the actual TTField shape expected in a patient.

Because the electrical impedance, resistance, resistivity, or conductivity of each organ of a patient modifies the TTField shape and a particular TTField shape may be needed to effectively target a tumor, new and improved assemblies and methods of using a physical 3D model to determine real-world interactions between the TTField and various organs are desired. It is to such assemblies and methods of producing and using the same, that the present disclosure is directed. The problem of using a physical 3D model to determine real-world interactions between the TTField and various organs is solved by a phantom, system and method, the phantom comprising: a container for housing a fluid and a conductive solution within a cavity defined by the container. The container has at least one exterior wall comprising an exterior surface and an interior surface defining the cavity, and the exterior wall is constructed of a non-conductive matrix material mixed with conductive particles to provide the exterior wall with an electrical impedance, resistance, resistivity, or conductivity to model electrical impedance, resistance, resistivity, or conductivity of a first biological component. A conductive solution is within the cavity. The conductive solution is at least one of a fluid solution, a suspension, and a gel and configured to model electrical impedance, resistance, resistivity, or conductivity of a second biological component. Optionally, one or more solid element within the cavity is configured to model electrical impedance, resistance, resistivity, or conductivity of one or more further biological component.

The problem of using a physical 3D model to determine real-world interactions between the TTField and various organs is further solved by the method, comprising: obtaining a 3-dimensional model of an object, the 3-dimensional model having a plurality of voxels, with each voxel provided with property information identifying or being usable to determine at least one of an impedance, a resistance, a resistivity, or a conductivity for the voxel; and using the 3-dimensional model to create a phantom, by creating one or more element defining at least a portion of the phantom and corresponding to voxels within the 3-dimensional model, at least one of the one or more element having a non-conductive matrix material with conductive particles dispersed therein to provide the at least one of the one or more element with an electrical impedance, resistance, resistivity, or conductivity corresponding to a predetermined first portion of the object.

An advantage of the current system is that the phantom has both solid and liquid/gel components. The solid components may be sufficiently rigid to form a container to house a liquid/gel, and can be selected such that the solid may be cast into a bowl shape, filled with a conductive solution (e.g., a conductive fluid or gel—such as a saline solution or hydrogel), and then allow a movable electric field probe to “travel” along the volume of the liquid component. For example, an all-gel phantom would require placing such a probe while curing the gel and its location becomes fixed. Further, an all-gel phantom dries out after a few hours which degrades its conductivity, whereas the solid outer container holding a solution and a gel component therein does not cause the gel to dry out, and the phantom can be used for many months. The current system also avoids diffusion of conductive particles between adjacent elements, which can be problematic in all-gel phantom systems.

The details of one or more implementations of the subject matter described in this specification are set forth in the accompanying drawings and the description below. Other aspects, features and advantages of the subject matter will become apparent from the description, the drawings, and the claims.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is an exemplary embodiment of a schematic diagram of electrodes as applied to living tissue.

FIG. 2 is an exemplary embodiment of a schematic diagram of an electronic device configured to generate a TTField.

FIG. 3A is a cross-sectional diagram of an exemplary embodiment of a phantom.

FIG. 3B is a cross-sectional diagram of another exemplary embodiment of a phantom.

FIG. 4A is a diagram of an exemplary embodiment of a first application system constructed in accordance with the present disclosure.

FIG. 4B is a diagram of an exemplary embodiment of a second application system constructed in accordance with the present disclosure.

FIG. 5 is a process flow diagram of an exemplary embodiment of a phantom creation process.

FIG. 6 is a process flow diagram of an exemplary embodiment of a transducer array placement process.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Headings are provided for convenience only and are not to be construed to limit the invention in any manner. Embodiments illustrated under any heading or in any portion of the disclosure may be combined with embodiments illustrated under the same or any other heading or other portion of the disclosure. Any combination of the elements described herein in all possible variations thereof is encompassed by the invention unless otherwise indicated herein or otherwise clearly contradicted by context.

Unless otherwise required by context, singular terms shall include pluralities and plural terms shall include the singular, with the exception that the term “plurality” as used herein, does not include the singular.

All patents or published patent applications referenced in any portion of this application are herein expressly incorporated by reference in their entirety to the same extent as if each individual patent or publication was specifically and individually indicated to be incorporated by reference.

All of the assemblies, systems, kits, and/or methods disclosed herein can be made and executed without undue experimentation in light of the present disclosure. Where a method claim does not specifically state in the claims or description that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” The term “plurality” refers to “two or more.”

The use of the term “at least one” will be understood to include one as well as any quantity more than one. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to collectively perform a task.

The term “resistance” refers to a degree to which a substance or device, or component of a device, opposes the passage of electric current causing energy dissipation. “Resistivity” is a fundamental property of a substance or material which refers to the degree to which the substance or material opposes the passage of electric current causing energy dissipation, but is standardized: a resistance per unit length and per unit of cross-sectional area at a specified temperature.

The term “impedance” refers to an effective resistance of an electric circuit or component to alternating current, arising from the combined effects of ohmic resistance and reactance.

The term “conductivity” refers to a degree to which a specified material conducts electricity, calculated as the ratio of the current density in the material to the electric field that causes the flow of current. The “conductivity” of a material is the reciprocal of the material's resistivity.

As used herein, the term “TTField” (TTFields, or TTF(s)) means tumor treating field. TTFields are low intensity (e.g., 1-4 V/cm) alternating electric fields of medium frequencies (about 50 kHz-1 MHz, and more preferably from about 50 kHz-500 kHz) that when applied to a conductive medium, such as a human body, via electrodes, may be used, for example, to treat tumors as described in U.S. Pat. Nos. 7,016,725, 7,089,054, 7,333,852, 7,565,205, 7,805,201, and 8,244,345 by Palti (each of which is incorporated herein by reference) and in a publication by Kirson (see Eilon D. Kirson, et al., “Disruption of Cancer Cell Replication by Alternating Electric Fields”, Cancer Res. 2004 64:3288-3295). TTFields have been shown to have the capability to specifically affect cancer cells and serve, among other uses, for treating cancer. TTFields therapy is an approved mono-treatment for recurrent glioblastoma (GBM), and an approved combination therapy with chemotherapy for newly diagnosed GBM patients.

As used herein, the term TTSignal(s) is an electrical signal that, when received by electrodes applied to a conductive medium, such as a human body, causes the electrodes to generate the TTField described above. The TT Signal is often an AC electrical signal.

The term “transducer array”, as used herein, may mean a conductive transducer array or a non-conductive transducer array. Exemplary transducer arrays may include, for example, transducer arrays disclosed in any one of U.S. Patent Publication No. 2021/0346693 entitled “CONDUCTIVE PAD GENERATING TUMOR TREATING FIELD AND METHODS OF PRODUCTION AND USE THEREOF” and U.S. Patent Publication No. 2022/0193404 A1 entitled “OPTIMIZATION OF COMPOSITE ELECTRODE” each of which are hereby incorporated herein in their entirety.

Turning now to the inventive concept(s), certain non-limiting embodiments thereof include a system and method of implementing the system, the system comprising a container for housing a fluid, the container having at least one exterior wall constructed of a non-conductive matrix material mixed with conductive particles to provide the exterior wall with an electrical impedance, resistance, resistivity, or conductivity to model electrical impedance, resistance, resistivity, or conductivity of a first biological component; and a conductive solution within the container, the conductive solution being configured to model electrical impedance, resistance, resistivity, or conductivity of a second biological component. In operation, the system may further comprise an electric field generator configured to generate an electrical signal (TTSignal) having an alternating current waveform at a frequency in a range from 50 kHz to 500 kHz; a first conductive lead electrically coupled to the electric field generator, the first conductive lead configured to carry the electrical signal to a transducer array electrically coupled to the first conductive lead, wherein the transducer array may be attached to the exterior wall of the container. Various aspects of the present disclosure are provided in detail below.

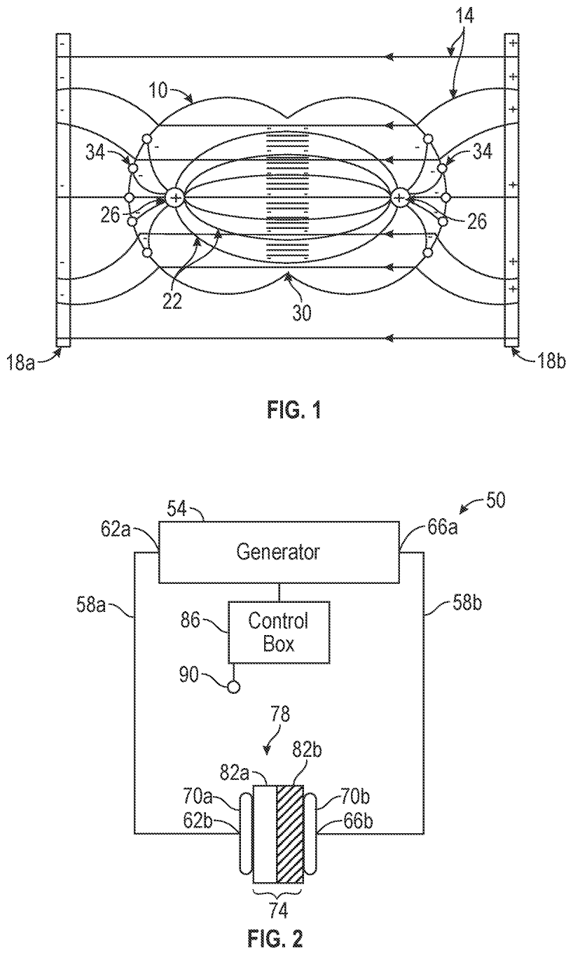

Referring now to the drawings and in particular to FIG. 1, shown therein is a diagram of an exemplary embodiment of a dividing cell 10, under the influence of external TTFields, generally indicated as lines 14, generated by a first electrode 18a having a negative charge and a second electrode 18b having a positive charge. Further shown are microtubules 22 that are known to have a very strong dipole moment. This strong polarization makes the microtubules 22, as well as other polar macromolecules and especially those that have a specific orientation within the cell 10 or its surroundings, susceptible to electric fields. The microtubules' 22 positive charges are located at two centrioles 26 while two sets of negative poles are at a center 30 of the dividing cell 10 and point of attachment 34 of the microtubules 22 to the cell membrane. The locations of the charges form sets of double dipoles and therefore are susceptible to electric fields of differing directions.

Turning now to FIG. 2, the TTFields described above that have been found to advantageously destroy tumor cells may be generated by an electronic apparatus 50. FIG. 2 is a simple schematic diagram of the electronic apparatus 50 illustrating major components thereof. The electronic apparatus 50 includes an electric field generator 54 and a pair of conductive leads 58, including first conductive lead 58a and second conductive lead 58b. The first conductive lead 58a includes a first end 62a and a second end 62b. The second conductive lead 58b includes a first end 66a and a second end 66b. The first end 62a of the first conductive lead 58a is conductively attached to the electric field generator 54 and the first end 66a of the second conductive lead 58b is conductively attached to the electric field generator 54.

The electric field generator 54 generates desirable electric signals (TTSignals) in the shape of waveforms or trains of pulses as an output. The second end 62b of the first conductive lead 58a is connected to a first transducer array 70a and the second end 66b of the second conductive lead 58b is connected to a second transducer array 70b, such that the first transducer array 70a and the second transducer array 70b are supplied with the electric signals (e.g., wave forms).

Each of the first transducer array 70a and the second transducer array 70b are in contact with, or otherwise associated with, a field target 74. The electric signals generate an electric field (i.e., TTField) that is capacitively coupled into the field target 74, the TTField having a frequency and an amplitude, to be generated between the first transducer array 70a and the second transducer array 70b in the field target 74. In one embodiment, the field target 74 is a phantom 78 generally comprising two or more conductive elements 82a-n shown in FIG. 2 as conductive element 82a and conductive element 82b, described in more detail below.

Each of the first transducer array 70a and the second transducer array 70b include one or more conductive electrode element that may be capacitively coupled with the field target 74 by a non-conductive layer. Alternative constructions for the first transducer array 70a and the second transducer array 70b may also be used, including, for example, transducer arrays using a non-conductive layer formed of a ceramic element that is disc shaped, or is not disc-shaped, and/or non-conductive layer(s) that use non-ceramic dielectric materials positioned over a plurality of flat conductors. Examples of the latter construct include polymer films disposed over electrical contacts on a printed circuit board or over flat pieces of metal.

In some embodiments, the first transducer array 70a and the second transducer array 70b may also include electrode elements that are not capacitively coupled with the field target 74. In this situation, each of the first transducer array 70a and the second transducer array 70b may be implemented using a region of a conductive material that is configured for placement against a person's body, with no insulating dielectric layer disposed between the conductive elements and the body. Examples of the conductive material include, but are not limited to, a conductive film, a conductive fabric, and/or a conductive foam. Other alternative constructions for implementing the first transducer array 70a and the second transducer array 70b may also be used, as long as they are capable of delivering TTFields to the field target 74. Optionally, a skin-contact layer may be disposed between the first transducer array 70a and the field target 74, and between the second transducer array 70b and the field target 74, in any of the embodiments described herein. The skin-contact layer helps to adhere/affix the first transducer array 70a and the second transducer array 70b to the field target 74, provides a conductive pathway for the electric fields to pass between the first and second transducer arrays 70a and 70b and the field target 74 through an intervening non-conductive or conductive layer, and is biocompatible. Examples of skin-contact layers include hydrogel as well as carbon conductive adhesive composites. The latter adhesives may comprise conductive particles, such as, for example, carbon black powder or carbon fibers, etc.

While the electronic apparatus 50 shown in FIG. 2 comprises only two transducer arrays 70 (the first transducer array 70a and the second transducer array 70b), yet, in some embodiments, the electronic apparatus 50 may comprise more than two transducer arrays 70.

The electric field generator 54 generates an alternating voltage wave form at frequencies in the range from about 50 kHz to about 1 MHz (preferably from about 100 kHz to about 500 kHz, or from about 100 kHz to about 300 kHz) (i.e., the TTFields). The required voltages are such that an electric field intensity in tissue within the treatment area is in the range of about 0.1 V/cm to about 10 V/cm, such as, for example, 1-4 V/cm. To achieve this field, the potential difference between two conductors 18 (not shown) of the first transducer array 70a and the second transducer array 70b is determined by the relative impedances of the system components, i.e., a fraction of the electric field on each component is given by that component's impedance divided by a total circuit impedance.

In order to optimize the electric field (i.e., TTField) distribution, the first transducer array 70a and the second transducer array 70b (pair of transducer arrays 70) may be configured or oriented differently depending upon the application in which the pair of transducer array 70a and 70b are to be used. The pair of transducer arrays 70a and 70b, as described herein, are externally applied to the field target 74. When the field target 74 is a patient, the pair of transducer arrays 70 may be applied to the patient's skin, in order to apply the electric current, and electric field (TTField), thereby generating current within the patient's tissue. Generally, the pair of transducer arrays 70 are placed on the patient's skin by a user (or helper) such that the electric field is generated across patient tissue within a treatment area. TTFields that are applied externally can be of a local type or widely distributed type, for example, the treatment of skin tumors and treatment of lesions close to the skin surface, or a tumor further in the body.

In one embodiment, the user may be a medical professional, such as a doctor, nurse, therapist, or other person acting under the instruction of a doctor, nurse, or therapist. In another embodiment, the user may be the patient, that is, the patient (and/or a helper) may place the transducer array 70a and the transducer array 70b on their treatment area.

Optionally and according to another exemplary embodiment, the electronic apparatus 50 includes a control box 86 and a temperature sensor 90 coupled to the control box 86, which are included to control the amplitude of the electric field so as not to generate excessive heating in the treatment area.

When the control box 86 is included, the control box 86 controls the output of the electric field generator 54, for example, causing the output to remain constant at a value preset by the user. Alternatively, the control box 86 sets the output at the maximal value that does not cause excessive heating of the treatment area. In either of the above cases, the control box 86 may issue a warning, or the like, when a temperature of the treatment area (as sensed by temperature sensor 90) exceeds a preset limit. The temperature sensor 90 may be mechanically connected to and/or otherwise associated with the first transducer array 70a or the second transducer array 70b so as to sense the temperature of the field target 74 at either one or both of the first transducer array 70a or the second transducer array 70b.

In one embodiment, the control box 86 may turn off, or decrease power of the TTSignal generated by the electrical field generator 54, if a temperature sensed by the temperature sensor 90 meets or exceeds a comfortability threshold. In one embodiment, the comfortability threshold is the temperature at which a patient would be made uncomfortable while using the transducer array 70a and the transducer array 70b. In one embodiment, the comfortability threshold is a temperature at or about 40 degrees Celsius. In one embodiment, the comfortability threshold is a temperature of between about 39 degrees Celsius and 42 degrees Celsius, or a specific selected temperature between about 39 degrees Celsius and 42 degrees Celsius, such as, for example, 41 degrees Celsius.

The conductive leads 58 are standard isolated conductors with a flexible metal shield, preferably grounded thereby preventing spread of any electric field generated by the conductive leads 58. The transducer array 70a and the transducer array 70b may have specific shapes and positioning so as to generate the TTField of a desired configuration, direction, and intensity at the treatment area and only at the treatment area so as to focus the treatment.

The specifications of the electronic apparatus 50 as a whole and its individual components are largely influenced by the fact that at the frequency of the TTFields living systems behave according to their “Ohmic”, rather than their dielectric properties.

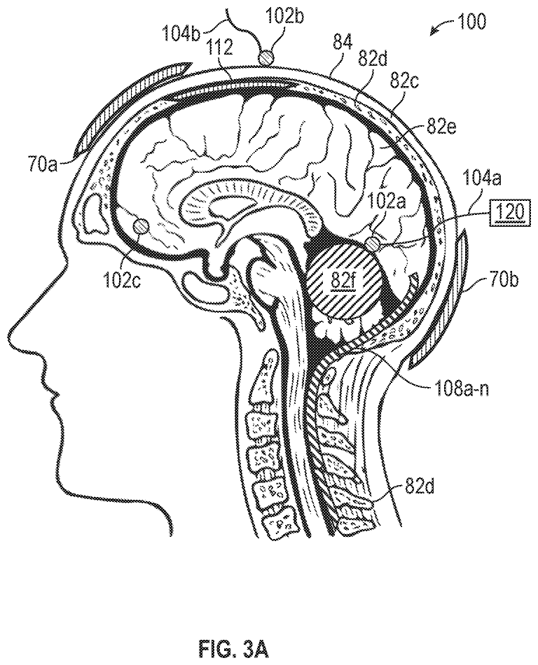

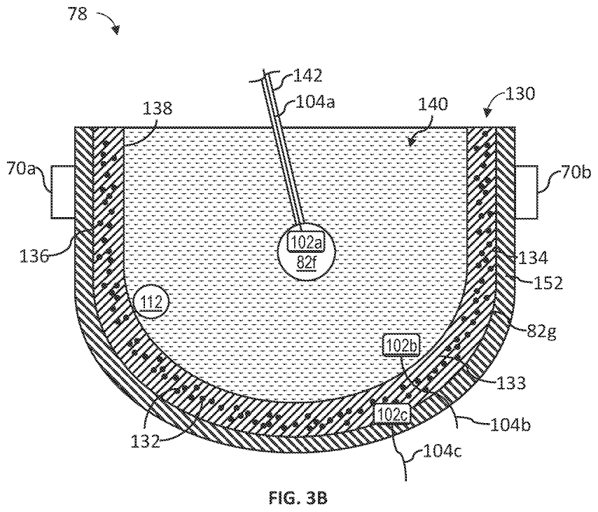

FIGS. 3A and 3B illustrate exemplary embodiments of phantoms 78 of FIG. 2. In some embodiments, the phantom 78 can be formed in the shape of human or non-human body parts, such as an arm, an elbow, a chest, a leg, a torso, and the like, or some combination thereof, or in the form of other types of objects, such as a cell phone, portion of a wall, or the like. In some embodiments, the phantom 78 is formed in the shape of a human body, or other animal body. In some embodiments, the phantom 78 is formed to be an anatomically-accurate representation of a particular human or other animal or a portion thereof.

Referring to FIG. 3A, shown therein is a cross-sectional diagram of an exemplary embodiment of the phantom 78 of FIG. 2 depicted as a phantom head 100 formed from a plurality of conductive elements 82a-n. In the example shown, the phantom head 100 is formed from a skin conductive element 82c, a bone conductive element 82d, and a brain conductive element 82e. The phantom head 100 shown in FIG. 3A is depicted as comprising three conductive elements 82c-e for simplicity only and may comprise any number of conductive elements 82a-n required by the user to appropriately model electrical impedance, resistance, resistivity, or conductivity of a selected portion of a human body, or a non-human body. Also shown in FIG. 3A is the first transducer array 70a and the second transducer array 70b on an outer surface 84 of the phantom head 100.

Therefore, in some embodiments, to appropriately model electrical impedance, resistance, resistivity, or conductivity of a selected biological component, the user should first determine a desired frequency, or range of frequencies, of a signal to be tested so appropriate electrical properties (e.g., impedance, resistance, resistivity, or conductivity) can be selected for the phantom 78 to best match the electrical properties of the selected biological component. The user may select one or more conductivity values from conductivity of biological components known in the art, such as from Gabriel, in The Dielectric Properties of Biological Tissues: II Measurements in the frequency range of 10 Hz to 20 GHz (Gabriel, S. et al., 1996, Phys. Med. Biol., 41, 2251).

In other embodiments, the user may select a mean conductivity for one or more biological component, such as calculated by Ramon et al. (Ramon C., Gargiulo P., Frõdgeirsson E. A. and Haueisen J. (2014) Changes in Scalp Potentials and Spatial Smoothing Effects of Inclusion of Dura Layer in Human Head Models for EEG Simulations; Front. Neuroeng. 7:32. doi:10.3389/fneng.2014.00032). For example only, and as calculated in Ramon, the user may select 1.35E⁻³ S/cm as a mean conductivity for the skin conductive element 82c, 6.25E⁻⁵ S/cm as a mean conductivity for the bone conductive element 82d, and 3.334E₋₃ S/cm as a mean conductivity for the brain conductive element 82e.

It should be noted that while the exemplary embodiment of the phantom 78 of FIG. 3A is depicted as being a hyper-accurate representation of a human head, the phantom 78 in some embodiments may be formed of a minimum number of conductive elements 82a-n required to model the field target 74 for a desired purpose.

By creating the phantom 78 (e.g., phantom 100 in FIG. 3A), users are able to determine actual values and shape of a TTField within and/or around the phantom 78 resulting from the application of an alternating electric field. For example, by utilizing one or more sensor 102a-n (discussed in more detail below in relation to FIG. 3B) users are able to determine a magnetic property, such as one or more electric field or electromagnetic field power/intensity; an electrical property such as a voltage (between spaced apart electrodes for example), a current, an inductance, a capacitance, and a resistance; a thermal property such as temperature; a pressure; a force; and/or the like at varying locations within the phantom 78.

In one embodiment, the determined actual values may be recorded for various configurations of the phantom 78 and utilized by the user to generate or improve computer simulations. In instances where the phantom 78 is representative of a specific patient, the determined actual values may be utilized to improve, or increase the therapeutic benefit of that specific patient's TTField therapy. Moreover, by applying the alternating electric field (e.g., a TTField) to the phantom 78, users are able to understand how alternating electric fields, such as the TTField, move through a human or non-human body and around various types of tissue and/or bone in an accurate, non-computer-simulation based setting.

In one embodiment, the phantom 78 comprises a conductive matrix material forming a container, and a conductive solution within the container. For example, the conductive matrix material may be a non-conductive polymer with conductive particles dispersed therein, and the conductive solution may be at least one of a fluid solution, a suspension, and a gel. Optionally, the phantom 78 may further comprise a solid component as described below in relation to FIG. 3B in more detail.

In the embodiment depicted in FIG. 3A, the phantom head 100 includes three conductive elements 82, the skin conductive element 82c, the bone conductive element 82d, and the brain conductive element 82e, with each of the conductive elements 82 bonded to at least a portion of another conductive element 82. The skin conductive element 82c includes a shape, thickness, and volume of human skin, and a substantially uniform electrical resistance, resistivity, impedance, and/or conductivity mimicking the electrical resistance, resistivity, impedance, and/or conductivity of human skin respectively. The bone conductive element 82d is positioned within the phantom 100 in a manner that mimics the location of bone within a human head. The bone conductive element 82d includes a shape, thickness, and volume of human bone within a human head, and may include a substantially uniform electrical resistance, resistivity, impedance, and/or conductivity mimicking the electrical resistance, resistivity, impedance, and/or conductivity of human bone respectively.

In one embodiment, the bone conductive element 82d is adjacent to and borders the skin conductive element 82c. The brain conductive element 82e includes a shape, thickness, and volume of a human brain. The brain conductive element 82e may include a substantially uniform electrical resistance, resistivity, impedance, and/or conductivity mimicking the electrical resistance, resistivity, impedance, and/or conductivity of the human brain respectively. The brain conductive element 82e is partially surrounded by and borders the bone conductive element 82d.

Although the phantom 78 is described by way of example in FIG. 3A as having three different types of conductive elements 82, i.e., the skin conductive element 82c, the bone conductive element 82d, and the brain conductive element 82e, the phantom 78 can be provided with other types of conductive elements, such as a blood vessel conductive element, a spinal fluid conductive element, a blood conductive element, a tumor conductive element, or the like. In some embodiments, the conductive elements 82 are connected together to form a continuous device having regions of varying electrical resistance, resistivity, impedance, and/or conductivity so as to collectively mimic the electrical resistance, resistivity, impedance, and/or conductivity, respectively, of a human head.

In one embodiment, the user may construct the phantom head 100 to be conductively similar to a human head, such as the head of the patient. That is, the user may construct the phantom head 100 such that the volume resistivity of the skin conductive element 82c is similar in volume resistivity to the skin of the patient, the volume resistivity of the bone conductive element 82d is similar in volume resistivity to the skull bone of the patient, and the volume resistivity of the brain conductive element 82e is similar in volume resistivity to the brain of the patient. In one embodiment, the user may also construct the phantom head 100 to include a target conductive element 82f having a volume resistivity similar in volume resistivity to the target, such as the target tumor.

In one embodiment, the user may construct the phantom head 100 to include one or more additional conductive element 82a-n modeling volume resistivity of other components in or around the patient's head, such as cartilage, eyes, hair, mucus, saliva, nerves, and the like. In one embodiment, the user may construct one or more conductive element 82a-n to simulate a portion of an organ, for example, the user may construct a first brain conductive element similar in volume resistivity to grey matter of the brain and a second brain conductive element similar in volume resistivity to white matter of the brain, or the user may construct a first bone conductive element similar in volume resistivity to bone marrow, a second bone conductive element similar in volume resistivity to spongy bone, and a third bone conductive element similar in volume resistivity to compact bone.

In one embodiment, the phantom head 100 may have one or more sensor 102a-n having a sensor lead 104a-n and associated with a particular location on or within the phantom head 100, such as a first sensor 102a having sensor lead 104a and associated with the target conductive element 82f and a second sensor 102b having sensor lead 104b and associated with the skin conductive element 82c. Additionally, each transducer array 70, such as the first transducer array 70a and the second transducer array 70b may include one or more sensor 102a-n. Each sensor 102a-n may include one or more of an electric field sensor, a voltage sensor, an ampere sensor, a temperature sensor, an electromagnetic field sensor, and/or the like. In one embodiment, by monitoring each sensor 102a-n, the user can determine an optimal placement of each of the one or more transducer array 70. The optimal placement of each of the one or more transducer array 70 may be determined by receiving data from the sensors 102a-n indicative of a maximized therapeutic benefit of the TTFields generated when one or more TTSignal is supplied to the first transducer array 70a, the second transducer array 70b, and any other transducer array 70 to be applied to the phantom 100.

In some embodiments, the one or more sensor 102a-n may be placed in a plurality of different locations throughout the phantom 78 (or 100). For example, in FIG. 3A the sensor 102c is placed in the frontal region of the brain conductive element 82e. By placing one or more sensor 102a-n through the phantom 100, the user may determine properties of the alternating electric field, e.g., TTField, at multiple locations within the phantom 100. In other embodiments, at least one of the one or more sensor 102a-n may be placed at an intersection between two or more conductive elements 82a-n. By placing a sensor 102 at the intersection between two or more conductive elements 82a-n, the user may determine one or more property of the alternating electric field as it passes from a first conductive element 82 to a second conductive element 82.

In some embodiments, each of the one or more sensor 102a-n includes a sensor lead 104a-n communicably coupled to an external device 120. By accessing the external device 120, the user may be able to determine a value for one or more property of each sensor 102a-n. In other embodiments, however, each of the one or more sensor 102a-n does not include the sensor lead 104a-n, and may include a wireless transceiver communicably coupled to the external device 120 using a wireless communication topology conforming to the requirements of Bluetooth, RFID, WIFI, Xbee, Z-wave, and the like, or some combination thereof, or any other wireless communication topology. In some embodiments, sensor 102 includes a sensor coupled to circuitry, such as a processor through an analog to digital converter, so as to provide digital signals that can be received, read, and interpreted by the processor. In these embodiments, the sensor lead may couple the processor to the wireless transceiver to permit the processor to forward data and instructions to the external device 120 via the wireless transceiver.

In one embodiment, the phantom head 100 may have one or more simulated vein 108a-n. While the one or more simulated vein 108a-n is referenced to as a vein, the one or more simulated vein 108a-n may also simulate an artery, or another part of the human body designed to carry or convey a model liquid. In one embodiment, each of the one or more simulated vein 108a-n may include a tube, hose, or the like operable to circulate the model liquid, such as blood, synthetic blood, and/or saline solution having electrical conductivity, impedance, resistance, and/or volume resistivity properties similar to human blood, within the phantom head 100. In one embodiment, the model liquid also has thermal conductivity properties similar to human blood. In one embodiment, the model liquid is circulated while receiving data from the one or more sensor 102a-n.

In one embodiment, the phantom 78 may be constructed to include one or more secondary element 112, such as a medical device or the one or more simulated vein 108a-n of FIG. 3A, for example. For example, if the phantom 78 is the phantom head 100, the user may construct the phantom head 100 to include one or more secondary element 112 that may be implanted in or placed on a patient's head, such as, for example, a medical device including a bone-anchored hearing aid, a cochlear implant, a metal plate such as one used to close a cranial defect, and/or the like. By constructing the phantom 78 to include the one or more secondary element 112, the user can measure changes in an electric field (e.g., TTField) within the phantom 78 due to the one or more secondary element 112.

In some embodiments, the one or more secondary element 112 is a medical device, such as a pace maker, that actively generates an electric field. In one embodiment, by constructing the phantom 78 as a chest cavity having one or more conductive element 82a-n with a volume resistivity similar to various organs within the chest cavity, and including the pace maker within the phantom 78, the user can measure the electric field due to the transducer arrays 70a-n as well as any fluctuations in the electric field caused by electric signals generated by the pace maker.

In one embodiment, one or more additional transducer array 70 (not shown) may be attached to the phantom head 100. The electric field generator 54, connected to each transducer array 70, may supply a first electrical signal having a first power and a first frequency to a first group of one or more transducer array 70, such as the first transducer array 70a and the second transducer array 70b, and supply a second electrical signal having a second power and a second frequency to a second group of one or more transducer arrays 70 attached to the phantom head 100 at the same instance in time. That is, the electric field generator 54, may simultaneously supply the first electric signal to the first group and the second electric signal to the second group. While the above embodiments describe only the first group and the second group, it is understood that there may be more than two groups. Further, in some embodiments, the electrical signals may not be simultaneously supplied.

FIG. 3B is a cross-sectional diagram of another exemplary embodiment of the phantom 78 of FIG. 2 depicted as a container 130 configured to model electrical impedance, resistance, volume resistivity, or conductivity of an exterior of a biological component, such as bone, and an interior of a biological component. The container 130 may be any shape including, but not limited to, cube, rectangular prism, sphere, cone, cylindrical, or any fanciful shape. The container 130 shown in FIG. 3B is a concave nearly hemispherical vessel having a plurality of conductive particles 132 disposed in the container walls thereof and formed to hold fluid, gel or other viscous substance, and/or solid. In one embodiment, the container 130 is modeled to depict a body part, e.g., the container 130 is modeled to form a skull configured to hold fluid or other viscous substance, and/or solid.

The container 130 includes at least one exterior wall 134 having an exterior surface 136 and an interior surface 138. The at least one exterior wall 134 may be formed as at least one conductive element 82. In some embodiments, the container 130 includes a single wall formed of a first conductive element 82g. The conductive element 82g may be configured to approximate and/or model electrical impedance, resistance, volume resistivity, or conductivity of an encapsulating component of a biological component (e.g., a skull). For example, the conductive element 82g may be configured to approximate and/or model electrical impedance, resistance, volume resistivity, or conductivity of a skull of a human body, or non-human body. When the container 130 is modeled to form a skull, the exterior wall 134 is provided with the electrical impedance, resistance, resistivity, or conductivity to model bone.

In one embodiment, the exterior wall 134, e.g., the conductive element 82g, comprises a matrix material 133 having a plurality of conductive particles 132 absorbed or adsorbed therein. The plurality of conductive particles 132 may include, for example, conductive carbon particles (such as conductive carbon fibers, conductive carbon granules, conductive carbon flakes, conductive graphite particles, conductive carbon black powder, conductive carbon nanoparticles, and conductive carbon nanotubes), conductive silver particles, conductive copper particles, and/or any other conductive metal particles or powder. A quantity of the plurality of conductive particles 132 may be adjusted to change electrical characteristics of the conductive element 82g such that the conductive element 82g is configured with electrical properties (e.g., impedance, resistance, volume resistivity, or conductivity) modeling electrical properties of a biological component. In one embodiment, the conductive particles 132 are homogeneously disposed within the matrix material 133 forming the exterior wall 134 and/or each conductive element 82. The conductive particles 132 may be sized such that, when disposed within the matrix material 133, the exterior wall 134 is solid and has an electrical conductivity, impedance, resistance or resistivity modeling the electrical conductivity, impedance, resistance or resistivity of the biological component.

In one embodiment, suitable quantities for the conductive particles 132 disposed in the matrix material 133 may range from 0.005-20.0%, or from 0.005-5.0%, or from 0.01-2.0%, or from 0.01-0.5%, or from 0.02-0.2% based on a solids of conductive particles to solids of the matrix material ratio (such as, for example, cured epoxy solids, or acrylic polymer solids).

In some embodiments, the matrix material 133 may be any non-conductive polymer. In some embodiments, the non-conductive polymer (matrix material 133) can form a desired shape (for example, by thermosetting or curing in a mold, or any other shape-forming technique). In one embodiment, the matrix material 133 is a multi-component epoxy material. In this embodiment, a conductive component, e.g., the plurality of conductive particles 132, may first be mixed with a first epoxy component to form a first conductive epoxy component. The first conductive epoxy component may then be mixed with a second epoxy component (for example, a hardener of a two-pack epoxy polymer system), to form a liquid epoxy. The liquid epoxy may then cure, thereby forming a solid conductive epoxy. In an embodiment, the curing process may occur in a mold, or undergo another shape forming process in order to be formed into a desired shape.

In one embodiment, the first epoxy component is a (liquid) epoxy resin which is mixed with conductive particles to form a conductive epoxy resin, and the second epoxy component is an epoxy hardener. The epoxy hardener, when added to the conductive epoxy resin, causes the conductive epoxy resin to begin a curing process into a cured conductive epoxy, i.e., a matrix material having conductive particles disposed therein.

The exterior wall 134 may thus be formed, for example, by pouring the liquid conductive epoxy into a mold before the liquid conductive epoxy has cured and, once the liquid conductive epoxy has cured into the cured conductive epoxy, e.g., the conductive element 82g, remove the cured conductive epoxy from the mold.

While the matrix material 133 is described herein as an epoxy material, the matrix material may be any castable matrix able to absorb or adsorb the conductive particles, such as an acrylic polymer (such as, for example, polymethyl methacrylate). In some embodiments, the matrix material 133 may be a rubber or an elastomer, such as, for example a silicone rubber or silicone elastomer. The matrix material 133 may be selected such that, after the matrix material 133, having the conductive particles 132 disposed therein, has been cast and set, the matrix material 133 is solid. Further, the matrix material 133 may limit or prevent diffusion of the conductive particles 132 from the matrix material 133 into a conductive solution, such as the conductive solution 140 described below. Additionally, the matrix material 133 may not lose particles to the atmosphere, thereby having continuous electrical properties providing an effective duration greater than 6 hours, e.g., the matrix material 133, may provide consistent electrical properties for longer than 6 hours. In some embodiments, the matrix material 133 provides consistent electrical properties for 7 days or longer, or for 6 months or longer.

The interior of the container 130 may be filled or partially filled with a conductive solution 140. The conductive solution 140 may be at least one of a fluid solution, a suspension, or a gel. The conductive solution 140 may be configured to approximate and/or model electrical impedance, resistance, volume resistivity, or conductivity of an interior of a biological component (e.g., brain matter, or blood, or organs, etc.). For example, the conductive solution 140 may be configured to approximate and/or model electrical impedance, resistance, volume resistivity, or conductivity of brain matter (i.e., white matter and/or gray matter). In some embodiments, the conductive solution 140 may be configured to approximate an average conductivity of white matter and gray matter, for example.

In some embodiments, the conductive solution 140 may be a saline solution, with salt content of the saline solution configured to approximate electrical properties (e.g., impedance, resistance, volume resistivity, or conductivity) of the interior of the biological component. In some embodiments, the conductive solution 140 may be a hydrogel, with a conductivity configured to approximate electrical conductivity of the interior of the biological component. In one embodiment, the hydrogel may be a conductive gel or semi-solid conductive gel. The hydrogel may be a modified hydrogel such as any hydrogel disclosed in detail in WIPO Patent Publication No. WO2021/226353 entitled “Conductive Pad Generating Tumor Treating Field and Methods of Production and Use Thereof”, which is hereby incorporated in its entirety.

Referring back to FIG. 3B, in some embodiments, one or more target conductive elements 82f may be formed on or attached to at least a portion of at least one movable probe 142. The target conductive element 82f may be configured to have a volume resistivity (or resistance, or impedance, or conductivity) approximating one or more target tumor. The target conductive element 82f may be positioned at any point on the movable probe 142. The at least one movable probe 142 having the target conductive element 82f formed thereon or attached thereto may be positioned about the interior of the container 130 and movable within the conductive solution 140 of the container 130.

For example, the at least one movable probe 142 may be positioned at a first site within the interior of the container 130 wherein one or more measurements may be obtained relative to the target conductive element 82f. The at least one movable probe 142 may then be positioned at a second site within the interior of the container 130 wherein one or more measurements may be obtained relative to the target conductive element 82f.

Additionally, in some embodiments, one or more target conductive elements 82f may be used with the at least one movable probe 142. To that end, the at least one movable probe 142 may be positioned at the first site wherein one or more measurements may be obtained relative to a first target conductive element 82f attached to the probe 142. The at least one movable probe 142 may be positioned at the second site wherein one or more measurements may be obtained relative to a second target conductive element 82f attached to the probe 142. It should be noted that additional probes, moveable or stationary, may be positioned within the interior of the container 130 in accordance with the present disclosure.

In some embodiments, the container 130, similar to the phantom head 100, may include one or more sensor 102a-n (shown in FIG. 3A), one or more simulated vein 108a-n (shown in FIG. 3A), and/or one or more secondary element 112 (shown in FIG. 3A) positioned with the interior of the container 130 (e.g., within the conductive solution 140 of the container 130). Similarly to FIG. 3A, the sensors 102a-n (e.g., 102a, 102b, 102c in FIG. 3B) may be connected to a sensor lead 104a-n (e.g., 104a, 104b, 104c in FIG. 3B), which, in turn, may be communicably coupled to an external device (such as 120, in FIG. 3A), and utilized similarly.

At least one movable probe 142 may be configured to be positioned within the container 130 to measure the electric field within the interior of the container 130. The interior of the container 130 is bounded by the interior surface 138 of the exterior wall 134. Referring to FIGS. 2 and 3B, the one or more transducer array 70 may be attached to the exterior surface 136 of the exterior wall 134 of the container 130 in the absence of a hydrogel layer 152 (discussed below). The electric field generator 54, connected to each transducer array 70, may supply a first electrical signal having a first power and a first frequency to a first group of one or more transducer array 70, such as the first transducer array 70a and the second transducer array 70b, and supply a second electrical signal having a second power and a second frequency to a second group of one or more transducer arrays 70 attached to the exterior of the container 130. The at least one movable probe 142, having at least one target conductive element 82f thereon or attached thereto, may be configured to measure the electric field within the interior container 130, and in some embodiments, within the target conductive element 82f during generation of the TTSignal from the electric field generator 54.

In some embodiments, the phantom 78 may include a hydrogel layer 152 on the exterior surface 136 of the exterior wall 134, wherein the hydrogel layer is configured to provide electrical impedance, resistance, resistivity, or conductivity to model electrical impedance, resistance, resistivity, or conductivity of skin. In this embodiment, the one or more transducer array 70 may be attached to the hydrogel layer 152 as shown for the first transducer arrays 70a and the second transducer array 70b in FIG. 3B. In other embodiments, the one or more transducer array 70 may be attached directly to the exterior surface 136 of the exterior wall 134 and at least partially within the hydrogel layer 152.

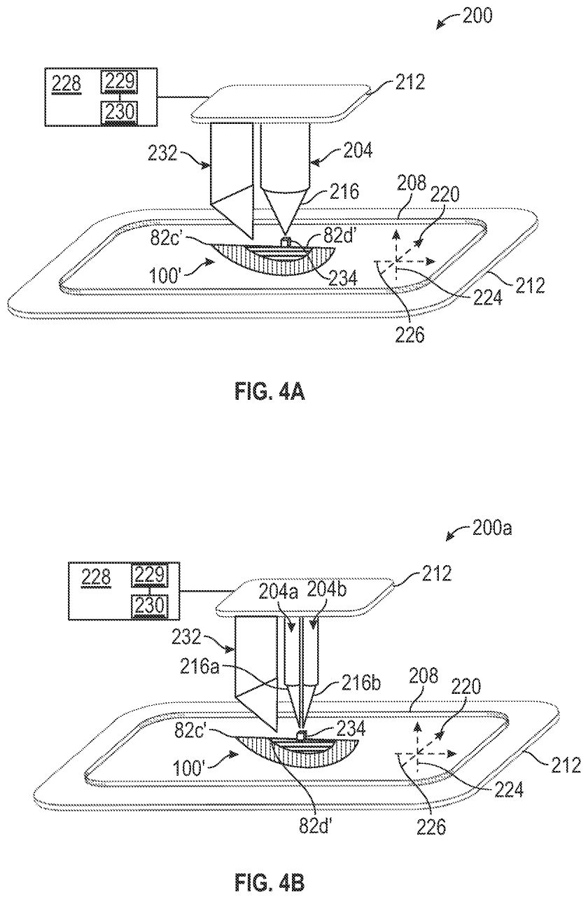

Referring now to FIG. 4A, shown therein is a diagram of an exemplary embodiment of a first application system 200 constructed in accordance with the present disclosure. The first application system 200 generally comprises one or more applicator 204 and a platform 208 moveably attached to a housing 212. Only one applicator 204 is shown for purposes of brevity; however, more than one applicator 204 may be utilized. The one or more applicator 204 further comprises at least a nozzle 216 to eject a conductive epoxy, described in more detail above, at an ejection rate. The platform 208 supports the phantom 78 while the phantom 78 is being constructed, depicted as partial phantom head 100′ having a partial skin conductive element 82c′ and a partial bone conductive element 82d′. In one embodiment, the first epoxy component, the second epoxy component, and the conductive component (e.g., in a liquid dispersion form) may be mixed within the applicator 204 and ejected as a liquid conductive epoxy from the nozzle 216 of the applicator 204. In one embodiment, the first epoxy component and the conductive component may be mixed at a first point in time forming a first conductive epoxy component. The first conductive epoxy component may then be mixed with the second epoxy component (e.g., hardener) within the applicator 204 and ejected as a liquid conductive epoxy.

In one embodiment, the applicator 204 may move in one of a first direction 220, a second direction 224, or a third direction 226, and combinations thereof. In one embodiment, the platform 208 may move in the first direction 220, the second direction 224, the third direction 226, or combination(s) thereof. The first direction 220 can be a y-direction, the second direction 224 can be a z-direction and the third direction 226 can be an x-direction. In one embodiment, the first application system 200 includes a controller 228 to control movement of the platform 208 and/or to control movement of the applicator 204.

In some embodiments, the controller 228 is loaded with a three-dimensional model of a proposed phantom having at least one proposed conductive element. In these embodiments, the three-dimensional model is provided with a plurality of voxels, with each voxel being a portion of one of the at least one proposed conductive element. Each of the voxels is provided with property information identifying (or used to determine) a particular impedance, resistance, resistivity, or conductance for the voxel. The property information is read by the controller 228 and can be used to create a voxel having the impedance, resistance, resistivity, or conductance.

In one embodiment, the controller 228 can be provided with circuitry, e.g., a memory 229, such as a non-transitory computer readable medium, communicably coupled to at least one processor 230. The memory 229 storing the three-dimensional model, and computer executable code configured to read the three-dimensional model, may be accessed by the processor 230. The processor 230, executing the computer executable code configured to read the three-dimensional model, may cause the applicator 204, or the platform 208, to move in one or more of the first direction 220, the second direction 224, or the third direction 226 and to cause the applicator 204 to eject the conductive epoxy at the ejection rate. In one embodiment, a computer system (not shown) is used to model the phantom 78 as a plurality of voxels of the three-dimensional model and to communicate the three-dimensional model to the controller 228 where the three-dimensional model may then be stored in the memory 229. In one embodiment, the controller 228 is in communication with the one or more computer system to receive the three-dimensional model or a plurality of voxels forming the three-dimensional model.

In one embodiment, the first application system 200 further includes a curing apparatus 232 to cause the first epoxy component, the second epoxy component, and the conductive component (in a liquid dispersion form) to cure more quickly into a three-dimensional conductive, set epoxy of the one or more conductive element 82. The curing apparatus 232 may supply a curing agent, such as heat, for example, to a particular voxel 234 comprising the mixed epoxy components in liquid form (e.g., the first epoxy component, the second epoxy component, and the conductive component). The curing apparatus 232, by applying the curing agent, thereby causes the particular voxel 234 of mixed epoxy components in liquid form to cure into a solid, or semi-solid, three-dimensional conductive epoxy forming a portion of the particular one of the one or more conductive element 82.

In one embodiment, for example, the user utilizes the controller 228 to cause the applicator 204 of the first application system 200 to eject a first liquid epoxy component (e.g., an epoxy resin), a second liquid epoxy component (e.g., an epoxy hardener), and a conductive component (e.g., conductive carbon) as a first mixed conductive epoxy having a first ratio, and the curing apparatus 232 to supply the curing agent (e.g., heat) to the mixed conductive epoxy for a first duration and at a first intensity to form a first voxel of the partial skin conductive element 82c′ having a volume resistivity (or resistance, or impedance, or conductivity) similar to the skin of the patient. The controller 228 may further cause the applicator 204 of the first application system 200 to eject a second mixed conductive epoxy having a second ratio, and may further cause the curing apparatus 232 to supply the curing agent to the second mixed conductive epoxy for a second duration and at a second intensity to form a second voxel of the partial bone conductive element 82d′ having a volume resistivity (or resistance, or impedance, or conductivity) similar to the skull bone of the patient.

In one embodiment, the controller 228, may slice the three-dimensional model into one or more layer wherein each layer is a plurality of substantially coplanar voxels and wherein each voxel of the plurality of substantially coplanar voxels corresponds to a volume of a model of a particular conductive element 82a-n. For each voxel on a particular layer, the controller 228 may cause the applicator 204 to eject a mixed conductive epoxy, comprised of at least a first epoxy component, a second epoxy component, and the conductive component, and to apply a curing agent to the voxel such that the voxel exhibits electrical conductivity, impedance, resistance and/or volume resistivity similar to the voxel's corresponding volume of the particular conductive element 82a-n, e.g., the resistivity of each voxel of the model is consistent with the resistivity of the voxel's corresponding volume of the particular conductive element 82a-n.

In one embodiment, the controller 228 causes all or most of the plurality of substantially coplanar voxels to be ejected and cured on a layer-by-layer basis, that is, if the controller 228 slices the three-dimensional model into a first layer having a first plurality of substantially coplanar voxels and a second layer having a second plurality of substantially coplanar voxels, the controller 228 may cause most or all of the first plurality of substantially coplanar voxels at the first layer to be formed before the controller 228 causes most or all of the second plurality of substantially coplanar voxels at the second layer to be formed. In one embodiment, the computer system, may slice the three-dimensional model into one or more layer where each layer is a plurality of substantially coplanar voxels and where each voxel of the plurality of substantially coplanar voxels corresponds to a volume of a model of a particular conductive element 82a-n.

In one embodiment, more than one applicator 204 may be used to form more than one phantom 78, or more than one portion of a phantom, simultaneously.

In one embodiment, one or more secondary element 112 (FIG. 3A), such as the one or more medical device, the one or more simulated vein 108a-n (FIG. 3A), and/or the like, may be placed on a particular layer of the partial phantom head 100′ as the partial phantom head 100′ is being constructed, or printed, by the first application system 200. By attaching the one or more secondary element 112 during construction of the phantom head 100, the mixed conductive epoxy may attach to the secondary element 112 when the mixed conductive epoxy is cured, thereby preventing movement of the one or more secondary element 112 as movement of the secondary element 112 may introduce errors into the phantom 78.

Referring now to FIG. 4B, shown therein is a diagram of an exemplary embodiment of a second application system 200a constructed in accordance with the present disclosure. The second application system 200a is similar in construction and function as the first application system 200 described above and shown in FIG. 4A except that the applicator 204 includes a first applicator 204a and a second applicator 204b wherein the first applicator 204a is operable to eject the first epoxy component at a first rate (in liquid form) through a first nozzle 216a and the second applicator 204b is operable to eject the second epoxy component at a second rate (in liquid form) through a second nozzle 216b to a same location to cause the first epoxy component, the second epoxy component, and a conductive component to mix and form a portion of one of the conductive element(s) 82.

In this embodiment, by adjusting the first ejection rate of the first epoxy component and the second ejection rate of the second epoxy component for a period of time, the user can select a ratio between the first epoxy component and the second epoxy component to adjust a cure rate. Additionally, an amount of conductive component in the mixed conductive epoxy may be adjusted to adjust a resulting impedance, resistance, resistivity, or conductivity of the mixed conductive epoxy once the mixed conductive epoxy is cured. By repeating the steps of applying the first epoxy component, second epoxy component, and conductive component, followed by curing the applied first epoxy component, second epoxy component, and conductive component, the second application system 200a can create the conductive elements 82a-n of the phantom 78.

The second application system 200a further comprises a platform 208 moveably attached to a housing 212. The platform 208 supports the phantom 78 while the phantom 78 is being constructed. In FIG. 4B, the phantom 78 is depicted as a partial phantom head 100′ having a partial skin conductive element 82c′ and a partial bone conductive element 82d′.

In some embodiments, the controller 228 is loaded with a three-dimensional model of a proposed phantom 78 having at least one proposed conductive element 82. In these embodiments, the three-dimensional model is provided with a plurality of voxels, with each voxel being a portion of one of the at least one proposed conductive element. Each of the voxels is provided with property information identifying (or used to determine) a particular conductive property such as impedance, resistance, resistivity, or conductivity for the voxel. The property information is read by the controller 228 and can be used to create a voxel having the conductive property, e.g., impedance, resistance, resistivity, and/or conductivity. The controller 228 can be provided with a memory 229, such as a non-transitory computer readable medium, communicably coupled to at least one processor 230. The memory 229 storing the three-dimensional model, and computer executable code configured to read the three-dimensional model, may be accessed by the processor 230. The processor 230, executing the computer executable code configured to read the three-dimensional model, may cause the first applicator 204a and the second applicator 204b, or the platform 208, to move in one or more of the first direction 220 (e.g., y-direction), the second direction 224 (e.g., z-direction), or the third direction 226 (e.g., x-direction) and to cause the first applicator 204a to eject the first epoxy component (in liquid form) at the first rate through the first nozzle 216a and the second applicator 204b is operable to eject the second epoxy component (in liquid form) at the second rate through the second nozzle 216b, where one or more of the first epoxy component and the second epoxy component includes, mixed therein, the conductive component at a particular ratio.

In one embodiment, a computer system (not shown) is used to model the phantom 78 as a plurality of voxels of the three-dimensional model and to communicate the three-dimensional model to the controller 228 where the three-dimensional model may then be stored in the memory 229. In one embodiment, the controller 228 is in communication with the one or more computer system to receive the three-dimensional model or a plurality of voxels forming the three-dimensional model. In one embodiment, the computer system may slice the three-dimensional model into one or more layer where each layer is a plurality of substantially coplanar voxels and where each voxel of the plurality of substantially coplanar voxels corresponds to a volume of a particular conductive element 82a-n. Other aspects of the second application system 200a are similar to those of first application system 200 described above. For example, the curing apparatus 232 may operate similarly (e.g., applying a curing agent to cure a voxel 234 of epoxy or mixed epoxy components), and the application system still has the ability to cause all or most of the plurality of substantially coplanar voxels to be ejected and cured on a layer-by-layer basis, as described above.

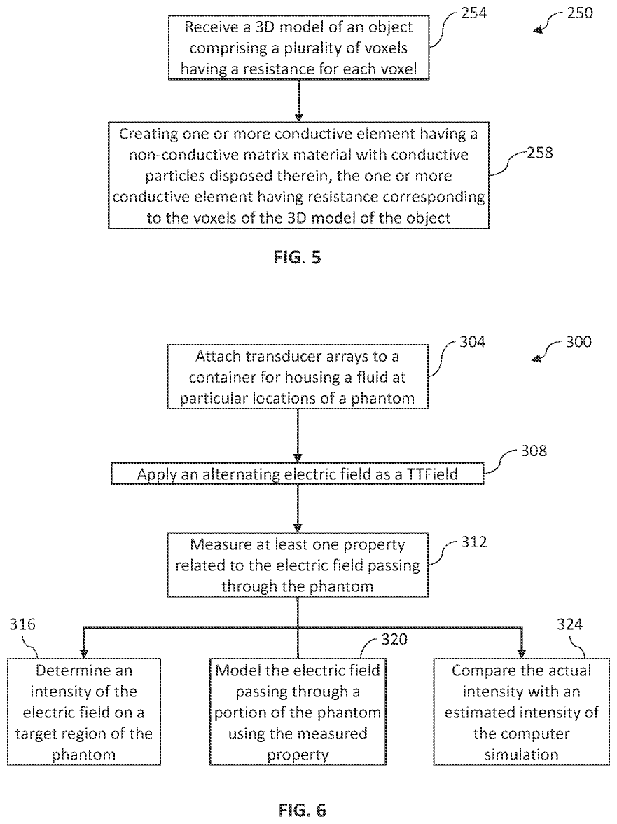

Referring now to FIG. 5, shown therein is an exemplary embodiment of a phantom creation process 250 generally comprising the steps of: receiving a model of an object (step 254), and creating one or more conductive element 82 having conductive particles 132, based at least in part on the model of the object (step 258).

In one embodiment, receiving a model of an object (step 254) includes receiving a 3D model of an object comprising a plurality of voxels having a resistance for each voxel. In some embodiments, the plurality of voxels includes one or more conductive property, such as an impedance, a resistance, a resistivity, and/or a conductance. In some embodiments, the object may be a 3D model (or a point cloud, etc.) of the phantom 78 or 100, or a predetermined portion of an object.

In one embodiment, receiving a model of an object (step 254) includes receiving the 3D model of an object comprising one or more predetermined portions where each predetermined portion corresponds to a particular conductive element 82. Each predetermined portion comprises a 3D model having a plurality of voxels having similar properties. For example, if the 3D model is of a head comprising a skull portion corresponding to a skull conductive element 82d and a brain portion corresponding to a brain conductive element 82e, each voxel of the skull portion may have similar first properties and each voxel of the brain portion may have similar second properties, where the first properties and the second properties are different.

In one embodiment, receiving a model of an object (step 254) includes receiving the 3D model of an object comprising a predetermined portion corresponding to one conductive element 82, where the 3D model of the predetermined portion is a model having a plurality of voxels having similar properties. For example, if the 3D model is of a head, the predetermined portion corresponding to the one conductive element may be a skull portion of the 3D model formed of a plurality of voxels having similar properties.

In one embodiment, creating one or more conductive element 82 with conductive particles 132 based at least in part on the model of the object (step 258) includes creating one or more conductive element 82 having a non-conductive matrix material 133 with conductive particles 132 disposed therein, the one or more conductive element 82 corresponding to the plurality of voxels.

In one embodiment, creating one or more conductive element with conductive particles 132, based at least in part on the model of the object (step 258) includes creating one or more conductive element 82 having a non-conductive matrix material 133 with conductive particles 132 disposed therein, the one or more conductive element 82 corresponding to the voxels of the 3D model of the object. In some embodiments, the non-conductive matrix material 133 is formed of an epoxy resin and a hardener. In some embodiments, the non-conductive matrix material 133 is formed of an acrylic polymer. In some embodiments, the non-conductive matrix material 133 is formed of a rubber or an elastomer, such as, for example a silicone rubber or silicone elastomer.

In one embodiment, creating one or more conductive element 82 with conductive particles 132 based at least in part on the model of the object (step 258) includes creating one or more conductive element 82 with conductive particles 132 disposed therein, where the conductive particles 132 are conductive carbon. In some embodiments, the conductive carbon 132 is mixed with an epoxy resin to form a conductive epoxy resin, which, when mixed with an epoxy hardener, forms the non-conductive matrix material 133 with conductive particles 132 disposed therein. In one embodiment, the conductive carbon is one or more of a plurality of carbon fibers, carbon flakes, carbon granules, graphite powder, or carbon black powder. In one embodiment, the conductive carbon is one or more of a plurality of conductive carbon nanoparticles and/or a plurality of conductive carbon nanotubes.

In one embodiment, mixing the conductive epoxy resin with the epoxy hardener occurs prior to creating the one or more conductive element 82 with conductive particles 132 based at least in part on the model of the object (step 258).

Referring now to FIG. 6, shown therein is an exemplary embodiment of a transducer array placement process 300 generally comprising the steps of: attaching transducer arrays 70 to the phantom 78 at particular locations (step 304), applying an alternating electric field to the phantom 78 with the transducer arrays 70 (step 308), measuring one or more sensor 102a-n to determine at least one field property (step 312), and performing at least one of the following steps (do one or more of): determining an intensity of the alternating electric field on a target region of the phantom 78 (step 316), modeling the electric field passing through a portion of the phantom 78 using the at least one measured property (step 320), and comparing the at least one field property to an estimated field property (step 324).

In one embodiment, attaching transducer arrays 70 to the phantom 78 at particular locations (step 304) may be performed by the user and may include attaching two or more transducer arrays 70 at particular first locations on the exterior wall 134 of the container 130 of the phantom 78 where the container 130 contains a conductive solution 140, the conductive solution 140 having a conductivity, and the exterior wall 134 of the container 130 comprising a matrix material 133 mixed with conductive particles 132 to provide an electrical conductivity of the exterior wall 134. In one embodiment, the electrical conductivity of the exterior wall 134 models an electrical conductivity of a first biological component of the phantom 78 and the electrical properties of the conductive solution 140 are configured to model electrical impedance, resistance, resistivity, and/or conductivity of a second biological component of the phantom 78.

In one embodiment, attaching transducer arrays 70 to the phantom 78 at particular locations (step 304) includes attaching transducer arrays 70 to the phantom 78 at particular pre-determined locations determined by a computer simulation.

In one embodiment, applying an alternating electric field to the phantom 78 with the transducer arrays 70 (step 308) includes applying an alternating electric field having a frequency in a range of from about 50 kHz to about 1 MHz (e.g., 100-500 kHz) and may include sending, by an electric field generator 54, a TTSignal to the transducer arrays 70 to generate a TTField within the phantom 78.

In one embodiment, applying an alternating electric field to the phantom 78 with the transducer arrays 70 (step 308) includes applying a TTField to the phantom 78 with the transducer arrays 70.

In one embodiment, applying an alternating electric field to the phantom 78 with the transducer arrays 70 (step 308) may include applying the alternating electric field for a first period of time, moving a probe 142 to a second location within the phantom 78, and applying the alternating electric field for a second period of time.

In one embodiment, measuring one or more sensor 102a-n to determine at least one field property (step 312) includes measuring at least one field property related to the alternating electric field passing through the phantom 78. In some embodiments, the at least one field property includes an intensity of the alternating electric field (e.g., a TTField) passing through at least a portion of the phantom 78 to obtain an actual electric field intensity as measured at the sensor 102a-n. In one embodiment, the portion of the phantom 78 may include a target conductive element, e.g., of a target treatment area.

In one embodiment, measuring one or more sensor 102a-n to determine at least one field property (step 312) includes measuring one or more sensor 102a-n associated with a particular location within the phantom 78 (e.g., attached to the moveable probe 142 moved to one or more particular location within the phantom 78).

In one embodiment, measuring one or more sensor 102a-n to determine at least one field property (step 312) includes measuring one or more of an alternating electric field strength, or intensity, a voltage, an amperage, or other electrical property, a magnetism or magnetic property, or a temperature, or some combination thereof. In one embodiment, the applied alternating electric field is the TTField and the target region is the target treatment area.

In one embodiment, measuring one or more sensor 102a-n to determine at least one field property (step 312) includes placing, or attaching, one or more of the plurality of sensors 102a-n on or within the container 130 of the phantom 78 and associating the one or more of the plurality of sensors 102a-n with a particular portion of the phantom 78. Each sensor 102a-n of the plurality of sensors 102a-n may provide at least one property.

In one embodiment determining an efficacy of the alternating electric field on a target region of the phantom 78 includes determining an intensity of the alternating electric field on a target region of the phantom 78 (step 316)

In one embodiment, the transducer array placement process 300 further includes the step of calculating a specific absorption rate of the alternating electric field by the phantom 78 based at least in part on the measured at least one field property related to the alternating electric field.

In one embodiment, the transducer array placement process 300 may be used to validate a simulation of TTField intensity (i.e., estimated electric field intensity, e.g., TTField intensity predicted within a computer model). The following may also be validated in lieu of or in addition to the TTField intensity: voltage, amperage, temperature, or some combination thereof in accordance with the present disclosure. In this embodiment, attaching transducer arrays 70 to the phantom 78 at particular locations (step 304) includes attaching transducer arrays 70 to the phantom 78 at particular locations determined by a computer simulation. The computer simulation may be one or more computer simulation to determine an estimated electric field intensity within a treatment area, e.g., an estimated TTField intensity. Generally, the computer simulation determines position(s) for electrodes or transducer arrays 70 for TTField treatment. The one or more computer model of electrical properties (e.g., impedance, resistance, resistivity, or conductivity) within the body (e.g., head, torso, other body part, etc.) may be generated by obtaining a CT scan and/or MRI image(s) of a particular portion of the patient's body. For example, disclosures within the following patents and patent publications detail segmentation of CT scans and/or MRI images to provide computer models of conductivity used to determine positions for electrodes in TTFields treatment: U.S. Pat. No. 10,188,851, filed on Oct. 27, 2016; U.S. Patent Publication No. 2020/0146586, filed on Nov. 12, 2019; and, U.S. Patent Publication No. 2020/0023179, filed on Jul. 18, 2019, which are all hereby incorporated by reference in their entirety. Within the computer simulation, an estimated electric field intensity may be determined for a treatment area (e.g., tumor) within the patient's body based on simulated positioning of the electrodes or transducer arrays.

In one embodiment, comparing the at least one field property to an estimated field property (step 324) includes comparing an estimated field intensity of the computer simulation (e.g., an estimated field property) and the actual field intensity of the phantom 78 (e.g., the at least one field property), to provide a resulting comparison output.

In one embodiment, comparing the at least one field property to an estimated field property (step 324) further includes validating the computer simulation of the estimated field intensity with the resulting comparison output. In other embodiments, the resulting comparison output may be used to update the computer simulation. For example, the resulting comparison output may validate the estimated field intensity provided by the computer simulation if the difference between the actual field intensity and the estimated field intensity is within a pre-determined threshold. In some embodiments, the resulting comparison may be used to adjust or calibrate the computer simulation (e.g., update one or more algorithms within the computer simulation).

### Non-Limiting Illustrative Embodiments

The following is a number list of non-limiting illustrative embodiments of the inventive concept disclosed herein:
Illustrative Embodiment 1. A phantom, comprising:
   a container for housing a fluid, the container having at least one exterior wall comprising an exterior surface and an interior surface defining a cavity, the exterior wall constructed of a non-conductive matrix material mixed with conductive particles to provide the exterior wall with an electrical impedance, resistance, resistivity, or conductivity to model electrical impedance, resistance, resistivity, or conductivity of a first biological component; and
   a conductive solution within the cavity, the conductive solution being at least one of a fluid solution, a suspension, and a gel and configured to model electrical impedance, resistance, resistivity, or conductivity of a second biological component.
Illustrative Embodiment 2. The phantom of Illustrative Embodiment 1, further comprising:
   one or more solid element within the cavity configured to model electrical impedance, resistance, resistivity, or conductivity of one or more further biological component.
Illustrative Embodiment 3. The phantom of Illustrative Embodiment 1, wherein the conductive solution is configured to model electrical impedance, resistance, resistivity, or conductivity of a brain, or a torso, or other body part.
Illustrative Embodiment 4. The phantom of Illustrative Embodiment 1, wherein the first biological component is a skull, and wherein the exterior wall is provided with the electrical impedance, resistance, resistivity, or conductivity to model bone.
Illustrative Embodiment 5. The phantom of Illustrative Embodiment 1, wherein the container has a shape corresponding to the first biological component.
Illustrative Embodiment 6. The phantom of Illustrative Embodiment 1, wherein the non-conductive matrix material is or comprises a polymer.
Illustrative Embodiment 7. The phantom of Illustrative Embodiment 1, wherein the non-conductive matrix material is or comprises an epoxy resin or an acrylic polymer.
Illustrative Embodiment 8. The phantom of Illustrative Embodiment 1, wherein the non-conductive matrix material is or comprises a rubber or an elastomer.
Illustrative Embodiment 9. The phantom of Illustrative Embodiment 1, wherein the conductive particles are conductive carbon particles.
Illustrative Embodiment 10. The phantom of Illustrative Embodiment 1, wherein the conductive particles are carbon fibers, carbon flakes, carbon granules, graphite powder, or carbon black powder.
Illustrative Embodiment 11. The phantom of Illustrative Embodiment 1, wherein the conductive particles are conductive carbon nanoparticles or conductive carbon nanotubes.
Illustrative Embodiment 12. The phantom of Illustrative Embodiment 1, wherein the conductive solution is a saline solution.
Illustrative Embodiment 13. The phantom of Illustrative Embodiment 1, wherein the conductive solution is a hydrogel.
Illustrative Embodiment 14. The phantom of Illustrative Embodiment 1, further comprising a hydrogel layer on the exterior surface 136 of the exterior wall 134, wherein the hydrogel layer is configured to provide electrical impedance, resistance, resistivity, or conductivity to model electrical impedance, resistance, resistivity, or conductivity of skin.
Illustrative Embodiment 15. The phantom of Illustrative Embodiment 1, wherein the one or more solid element within the cavity is configured to provide electrical impedance, resistance, resistivity, or conductivity to model electrical impedance, resistance, resistivity, or conductivity of a tumor.
Illustrative Embodiment 16. The phantom of Illustrative Embodiment 15, wherein the solid element is formed on, or attached to, a movable probe, which may be positioned within the cavity and is movable within the conductive solution of the container.
Illustrative Embodiment 17. A method, comprising:
   obtaining a 3-dimensional model of an object, the 3-dimensional model having a plurality of voxels, with each voxel provided with property information identifying or being usable to determine at least one of an impedance, a resistance, a resistivity, ora conductivity for the voxel; and
   using the 3-dimensional model to create a phantom, by creating one or more element defining at least a portion of the phantom and corresponding to voxels within the 3-dimensional model, at least one of the one or more element having a non-conductive matrix material with conductive particles dispersed therein to provide the at least one of the one or more element with an electrical impedance, resistance, resistivity, or conductivity corresponding to a predetermined first portion of the object.
Illustrative Embodiment 18. The method of Illustrative Embodiment 17, wherein the non-conductive matrix material is a multi-component epoxy material formed of an epoxy resin and a hardener, and wherein the method comprises mixing the conductive particles with the epoxy resin to form a conductive epoxy resin, and mixing the conductive epoxy resin with the hardener.
Illustrative Embodiment 19. The method of Illustrative Embodiment 17, wherein the conductive particles are conductive carbon particles, and further comprising mixing conductive carbon particles with an epoxy resin to form a conductive epoxy resin.
Illustrative Embodiment 20. The method of Illustrative Embodiment 19, further comprising mixing the conductive epoxy resin with a hardener.
Illustrative Embodiment 21. The method of Illustrative Embodiment 20, wherein mixing the conductive epoxy resin with the hardener occurs prior to creating the phantom with the 3-dimensional model.
Illustrative Embodiment 22. The method of Illustrative Embodiment 17, wherein at least one of the one or more element is a conductive solution being a fluid solution, a suspension, or a gel and configured to model an electrical impedance, resistance, resistivity, or conductivity corresponding to a predetermined second portion of the object.
Illustrative Embodiment 23. The method of Illustrative Embodiment 17, wherein at least one of the one or more element is a solid element configured to model an electrical impedance, resistance, resistivity, or conductivity corresponding to a predetermined third portion of the object.
Illustrative Embodiment 24. The method of Illustrative Embodiment 17, wherein at least one of the one or more element is a solid element configured to model an electrical impedance, resistance, resistivity, or conductivity corresponding to a tumor.
Illustrative Embodiment 25. The method of Illustrative Embodiment 24, wherein at least one of the one or more element is a conductive solution being a fluid solution, a suspension, or a gel and configured to model an electrical impedance, resistance, resistivity, or conductivity corresponding to a predetermined second portion of the object and wherein the solid element is formed on, or attached to, a movable probe, which may be positioned within the exterior wall of the phantom and is movable within the conductive solution of the phantom.
Illustrative Embodiment 26. A method, comprising:
   attaching transducer arrays to a phantom on an exterior wall of a container for housing a conductive solution, at particular locations on the phantom, the container containing the conductive solution, the exterior wall of the container comprising a matrix material with conductive particles dispersed therein to provide the exterior wall with an electrical impedance, resistance, resistivity, or conductivity to model electrical impedance, resistance, resistivity, or conductivity of a first biological component, the conductive solution being a fluid solution, a suspension, or a gel and configured to model electrical impedance, resistance, resistivity, or conductivity of a second biological component;
   applying an alternating electric field to the phantom via the transducer arrays;
   measuring at least one property related to the alternating electric field passing through at least a portion of the phantom with a plurality of sensors; and
   performing at least one of the following steps:
   determining an intensity of the alternating electric field at a target region within the phantom; and
   modeling the alternating electric field passing through the phantom using data measured by the plurality of sensors.
Illustrative Embodiment 27. The method of Illustrative Embodiment 26, wherein applying the alternating electric field includes applying a tumor treating field to the phantom via the transducer arrays.
Illustrative Embodiment 28. The method of Illustrative Embodiment 26 further comprising calculating a specific absorption rate of the alternating electric field by the phantom based at least in part on the measured at least one property related to the alternating electric field.
Illustrative Embodiment 29. The method of Illustrative Embodiment 26 further comprising attaching the plurality of sensors on or within the container of the phantom, each of the sensors associated with a particular portion of the phantom.
Illustrative Embodiment 30. The method of Illustrative Embodiment 29 wherein measuring at least one property related to the alternating electric field further includes obtaining a measurement from at least one of the plurality of sensors to determine the at least one property.
Illustrative Embodiment 31. The method of Illustrative Embodiment 30 wherein measuring the at least one property related to the alternating electric field further includes obtaining a measurement from at least one of the plurality of sensors to determine a temperature related to the alternating electric field passing through the particular portion of the phantom.
Illustrative Embodiment 32. The method of Illustrative Embodiment 30 wherein measuring the at least one property related to the alternating electric field further includes obtaining a measurement from at least one of the plurality of sensors to determine an electrical property related to the alternating electric field passing through the phantom.
Illustrative Embodiment 33. The method of Illustrative Embodiment 30 wherein measuring the at least one property related to the alternating electric field further includes obtaining a measurement from at least one of the plurality of sensors to determine a magnetic property related to the alternating electric field passing through the phantom.
Illustrative Embodiment 34. The method of Illustrative Embodiment 30, wherein applying the alternating electric field includes applying a tumor treating field to the phantom via the transducer arrays.
Illustrative Embodiment 35. The method of Illustrative Embodiment 34, wherein modeling the tumor treating field includes determining the intensity of the alternating electric field on the target region within the phantom.
Illustrative Embodiment 36. A method, comprising:
   attaching transducer arrays to a phantom on an exterior wall of a container for housing a conductive solution, at pre-determined locations based on a computer simulation, the container containing the conductive solution, the exterior wall of the container comprising a matrix material with conductive particles dispersed therein to provide the exterior wall with an electrical impedance, resistance, resistivity, or conductivity to model electrical impedance, resistance, resistivity, or conductivity of a first biological component, the conductive solution being at least one of a fluid solution, a suspension, or a gel and configured to model electrical impedance, resistance, resistivity, or conductivity of a second biological component;
   applying an alternating electric field to the phantom via the transducer arrays;
   measuring at least one property related to the alternating electric field passing through at least a portion of the phantom to obtain an actual electric field intensity; and,
   comparing the actual electric field intensity to an estimated electric field intensity obtained from the computer simulation.

It is contemplated that all embodiments of the Apparatus, such as the phantom constructs as described herein, may be considered in combination with the described methods of the invention. Unless clearly contradicted by the description, all variations and combinations of the elements described for one embodiment, are herein disclosed as variations and combinations of the elements for other embodiments described herein, both in descriptions of Apparatus, and for descriptions of methods, as well as Apparatus(es) included in descriptions of the methods(s).

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. A phantom, comprising:
a container for housing a fluid, the container having at least one exterior wall comprising an exterior surface and an interior surface defining a cavity, the exterior wall constructed of a non-conductive matrix material mixed with conductive particles to provide the exterior wall with an electrical impedance, resistance, resistivity, or conductivity to model electrical impedance, resistance, resistivity, or conductivity of a first biological component; and
a conductive solution within the cavity, the conductive solution being at least one of a fluid solution, a suspension, and a gel and configured to model electrical impedance, resistance, resistivity, or conductivity of a second biological component.

2. The phantom of claim 1, further comprising:
one or more solid element within the cavity configured to model electrical impedance, resistance, resistivity, or conductivity of one or more further biological component.

3. The phantom of claim 1, wherein the conductive solution is configured to model electrical impedance, resistance, resistivity, or conductivity of a brain, or a torso, or other body part.

4. The phantom of claim 1, wherein the first biological component is a skull, and wherein the exterior wall is provided with the electrical impedance, resistance, resistivity, or conductivity to model bone.

5. The phantom of claim 1, wherein the non-conductive matrix material is or comprises a polymer.

6. The phantom of claim 1, wherein the non-conductive matrix material is or comprises an epoxy resin or an acrylic polymer.

7. The phantom of claim 1, wherein the non-conductive matrix material is or comprises a rubber or an elastomer.

8. The phantom of claim 1, wherein the conductive particles are conductive carbon particles.

9. The phantom of claim 1, wherein the conductive particles are carbon fibers, carbon flakes, carbon granules, graphite powder, or carbon black powder.

10. The phantom of claim 1, wherein the conductive particles are conductive carbon nanoparticles or conductive carbon nanotubes.

11. The phantom of claim 1, wherein the conductive solution is a saline solution.

12. The phantom of claim 1, wherein the conductive solution is a hydrogel.

13. The phantom of claim 1, further comprising a hydrogel layer on the exterior surface of the exterior wall, wherein the hydrogel layer is configured to provide electrical impedance, resistance, resistivity, or conductivity to model electrical impedance, resistance, resistivity, or conductivity of skin.

14. The phantom of claim 2, wherein the one or more solid element within the cavity is configured to provide electrical impedance, resistance, resistivity, or conductivity to model electrical impedance, resistance, resistivity, or conductivity of a tumor.

15. The phantom of claim 14, wherein the one or more solid element is formed on, or attached to, a movable probe, which may be positioned within the cavity and is movable within the conductive solution of the container.

16. A method, comprising:
attaching transducer arrays to a phantom on an exterior wall of a container for housing a conductive solution, at particular locations on the phantom, the container containing the conductive solution, the exterior wall of the container comprising a matrix material with conductive particles dispersed therein to provide the exterior wall with an electrical impedance, resistance, resistivity, or conductivity to model electrical impedance, resistance, resistivity, or conductivity of a first biological component, the conductive solution being a fluid solution, or a suspension, or a gel and configured to model electrical impedance, resistance, resistivity, or conductivity of a second biological component;
applying an alternating electric field to the phantom via the transducer arrays;
measuring at least one property related to the alternating electric field passing through at least a portion of the phantom with a plurality of sensors; and
performing at least one of the following steps:
determining an intensity of the alternating electric field at a target region within the phantom; and
modeling the alternating electric field passing through the phantom using data measured by the plurality of sensors.

17. The method of claim 16, wherein applying the alternating electric field includes applying a tumor treating field to the phantom via the transducer arrays.

18. The method of claim 16 further comprising attaching the plurality of sensors on or within the container of the phantom, each of the sensors associated with a particular portion of the phantom, and wherein measuring at least one property related to the alternating electric field further includes obtaining a measurement from at least one of the plurality of sensors to determine the at least one property.

19. The method of claim 18 wherein measuring the at least one property related to the alternating electric field further includes obtaining a measurement from at least one of the plurality of sensors to determine an electrical property or a magnetic property related to the alternating electric field passing through the phantom.

20. A method, comprising:
attaching transducer arrays to a phantom on an exterior wall of a container for housing a conductive solution, at pre-determined locations based on a computer simulation, the container containing the conductive solution, the exterior wall of the container comprising a matrix material with conductive particles dispersed therein to provide the exterior wall with an electrical impedance, resistance, resistivity, or conductivity to model electrical impedance, resistance, resistivity, or conductivity of a first biological component, the conductive solution being at least one of a fluid solution, or a suspension, or a gel and configured to model electrical impedance, resistance, resistivity, or conductivity of a second biological component;
applying an alternating electric field to the phantom via the transducer arrays;
measuring at least one property related to the alternating electric field passing through at least a portion of the phantom to obtain an actual electric field intensity; and,
comparing the actual electric field intensity to an estimated electric field intensity obtained from the computer simulation.
